(19) United States
(12) Patent Application Publication
(10) Pub. No.: US 2025/0333438 A1
(43) Pub. Date: Oct. 30, 2025

(54) INJECTION ELUTION METHODS FOR AFFINITY CHROMATOGRAPHY

(71) Applicant: Waters Technologies Corporation, Milford, MA (US)

(72) Inventors: Beatrice Muriithi, Attleboro, MA (US); Kevin Wyndham, Worcester, MA (US); Martin Gilar, Franklin, MA (US); Nathan Canniff, Boston, MA (US)

(73) Assignee: Waters Technologies Corporation, Milford, MA (US)

(21) Appl. No.: 19/189,889

(22) Filed: Apr. 25, 2025

Related U.S. Application Data
(60) Provisional application No. 63/639,317, filed on Apr. 26, 2024.

(51) Int. Cl. C07K 1/22; C12N 15/10; G01N 30/88

(52) U.S. Cl. CPC C07K 1/22; C12N 15/101; G01N 30/88; G01N 2030/8827; G01N 2030/8831

(57) ABSTRACT

The present disclosure is directed to methods of performing affinity chromatography. The injection elution methods disclosed herein afford rapid and robust elution of a target analyte from an affinity chromatography column, therefore improving workflow efficiency. Further, the single injection elution methods result in highly concentrated samples, mitigating the need for sample concentration or manipulation.

### CROSS REFERENCE TO RELATED APPLICATIONS

This application claims priority to and the benefit of U.S. Provisional Application Ser. No. 63/639,317, filed Apr. 26, 2024, and entitled “Injection Elution Methods for Affinity Chromatography”. The contents of the foregoing application are incorporated herein by reference in their entirety.

### FIELD OF TECHNOLOGY

The present disclosure relates generally to affinity chromatography methods, in particular methods for eluting samples from affinity chromatography columns.

### BACKGROUND

Affinity chromatography methods are pervasive in the pharmaceutical, biotechnology, and chemical industries, and can be used to purify and isolate analytes of interest from heterogenous samples. Affinity chromatography relies on a functionalized stationary phase that selectively binds the target analyte. The target analyte can then be eluted from the stationary phase using an elution buffer mobile phase, resulting in a purified sample of the target analyte.

Typically, elution of the target analyte utilizes a gradient elution method, wherein the elution buffer is introduced into the mobile phase over a length of time. Gradient elution methods can require long elution times and may result in broad peaks, affecting the volume and concentration of your eluted sample. The longer elution times and diluted sample concentrations that are produced by gradient elution methods can impact downstream analytical processes and overall efficiency. Therefore, a need in the art exists for new elution methods in affinity chromatography.

### SUMMARY OF TECHNOLOGY

Disclosed herein are single injection elution methods for use with affinity chromatography techniques. In one aspect, the methods of the technology use affinity chromatography columns having a stationary phase comprising nonporous polymer particles typically having an average particle size between 1.0 μm to 10 μm and a functionalized surface. The injection elution methods (also referred to as single injection elution methods) of the present disclosure utilize single injections of elution buffer at small volumes, ranging from about 1 μL to about 50 μL, that result in high recovery levels of a target analyte with minimal dilution of sample concentration. Further, the injection elution of the present technology is rapid, resulting in elution times of less than 2 minutes.

Accordingly, in one aspect disclosed herein is a method of purifying a target analyte, the method comprising loading a sample comprising the target analyte onto an affinity chromatography column, washing the affinity chromatography column with a wash buffer, and eluting the target analyte from the affinity chromatography column using a single injection of an elution buffer, the single injection having a volume of between about 1 μL to about 50 μL. In other embodiments, the single injection has a volume between 50-100% of the column volume of the affinity chromatography column. The affinity chromatography column comprises a plurality of nonporous polymer particles, wherein each particle within the plurality of nonporous polymer particles includes a polymer core and a hydrophilic surface on an outer layer of the polymer core. In some embodiments, the affinity chromatography column comprises a plurality of nonporous polymer particles and one or more affinity agents conjugated to the particle. In some embodiments, the one or more affinity agents are conjugated directly to a surface of the nonporous polymer particle. In some embodiments, the one or more affinity agents are conjugated indirectly to a surface of the nonporous polymer particle. The indirect conjugation may be via a linker group, or in preferred embodiments via an interaction with one or more streptavidin molecules on a surface of each particle within the plurality of nonporous polymer particles. Typically, the nonporous polymer particles have an average particle size between 1.0 μm to 10 μm.

In some embodiments, the affinity agent is an immunoglobulin-binding protein, an antibody or antigen-binding fragment thereof, or an oligonucleotide. In some embodiments, the affinity agent is biotinylated. In some embodiments, the immunoglobulin-binding protein is Protein A, Protein G, Protein A/G, Protein L, or a binding domain thereof. In some embodiments, the antibody or antigen-binding fragment thereof binds to insulin, an AAV capsid, tacrolimus, troponin, IgG, a cytokine, a dsRNA, a host cell protein, or perfluoroalkyl substances (PFAS). In some embodiments, the AAV capsid is AAV1, AAV2, AAV3, AAV4, AAV5, AAV6, AAV7, AAV8, AAV9, AAVrh10, or a synthetic serotype thereof. In some embodiments, the oligonucleotide is a poly-T oligonucleotide.

In some embodiments, the wash buffer comprises sodium phosphate (i.e., PBS). In some embodiments, the wash buffer includes ammonium acetate, ammonium formate, or sodium chloride. In some embodiments, the wash buffer has a pH of between 6.0 to 8.0.

In some embodiments, the elution buffer comprises hydrochloric acid, trifluoroacetic acid, difluoroacetic acid, formic acid, acetic acid, or phosphoric acid. In some embodiments, the elution buffer has a pH of between 0.9 to 3.5. In some embodiments, the elution buffer is water. In some embodiments, the elution buffer comprises 0.1-5.0% DMSO. In some embodiments, the elution buffer comprises 1% DMSO.

In some embodiments, the single injection has a volume of about 1 μL, about 10 μL, about 20 μL, about 30 μL, about 40 μL, or about 50 μL.

In some embodiments, the affinity chromatography column is connected to a high-performance liquid chromatography (HPLC) system, ultra-high performance liquid chromatography (UHPLC) system, or fast protein liquid chromatography (FPLC) system.

The methods disclosed herein may further comprise a step of detecting the target analyte with a detector. In some embodiments, the detector is an ultraviolet spectroscopy detector, a fluorescence spectroscopy detector, and/or a mass spectrometry detector.

In some embodiments, the eluting step is performed in less than 2 minutes. In some embodiments, the eluting step is performed in less than 1 minute. The eluting step may be performed in less than 30 seconds, less than 20 seconds, or less than 10 seconds. In some embodiments, the eluting step results in a peak width of between 1 to 10 seconds. In some embodiments, the eluting step is repeated, including with the same volume or a different volume as the first injection.

The eluting step may result in at least 50% recovery of the target analyte. In some embodiments, the eluting step may result in at least 70% recovery of the target analyte.

### BRIEF DESCRIPTION OF THE DRAWINGS

The technology will be more fully understood from the following detailed description taken in conjunction with the accompanying drawings, in which:

FIGS. 1A-1C depict the purification of a monoclonal antibody using the single injection elution methods described herein. FIG. 1A provides a chromatogram following loading of a sample but prior to elution. FIG. 1B provides a chromatogram following the elution of the sample using the single injection elution method. FIG. 1C provides a comparison of peak volumes following elution with different volumes of elution buffer.

FIGS. 2A-2B compare the purification of a monoclonal antibody using single injection elution and gradient elution methods. FIG. 2A provides a chromatogram of sample eluted using the single injection elution methods described herein. FIG. 2B provides a chromatogram of a sample eluted using gradient elution methods on columns having nonporous particles.

FIGS. 3A-3B demonstrate the linear correlation between sample concentration and peak area using the single injection elution methods disclosed herein. FIG. 3A provides chromatograms of the eluted samples. FIG. 3B depicts the linear correlation of the chromatograms of FIG. 3A, with the x-axis being sample concentration and the y-axis being peak area.

FIGS. 4A-4J provide chromatograms comparing single injection elution and gradient elution methods for various MS-compatible elution buffers. FIG. 4A (single injection elution) and FIG. 4B (gradient elution) provide chromatograms for a formic acid elution buffer. FIG. 4C (single injection elution) and FIG. 4D (gradient elution) provide chromatograms for a difluoroacetic acid buffer. FIG. 4E (single injection elution) and FIG. 4F (gradient elution) provide chromatograms for a trifluoroacetic acid elution buffer. FIG. 4G (single injection elution) and FIG. 4H (gradient elution) provide chromatograms for a phosphoric acid elution buffer. FIG. 4I (single injection elution) and FIG. 4J (gradient elution) provide chromatograms for a hydrochloric acid elution buffer.

FIGS. 5A-5B demonstrate the purification of AAV9 capsids using the single injection elution methods provided herein. FIG. 5A provides a chromatogram showing that the column was loaded to saturation. FIG. 5B provides a chromatogram of sample eluted using single injections of elution buffer.

FIG. 6A-6C demonstrate the purification of Firefly luciferase (FLuc) mRNA using single injection elution and gradient elution methods. FIG. 6A provides a chromatogram of FLuc mRNA eluted using a gradient elution. FIG. 6B provides a chromatogram of FLuc mRNA using a single injection elution disclosed herein. FIG. 6C compares the percent recovery of the FLuc mRNA for different single injection elution volumes.

FIG. 7A-7C demonstrate the purification of double-stranded RNA (dsRNA) using single injection elution methods. FIG. 7A provides a chromatogram of dsRNA eluted using a single injection elution method with water. FIG. 7B provides chromatograms of different concentrations of dsRNA using a single injection elution method with water. FIG. 7C shows the linear correlation between peak area and dsRNA concentration of the chromatograms of FIG. 7B. FIG. 7D provides chromatograms of different concentrations of dsRNA using a single injection elution method with 1% DMSO in water. FIG. 7E shows the linear correlation between peak area and dsRNA concentration of the chromatograms of FIG. 7D.

FIG. 8A-8B demonstrate the impact of interacting with a chromatography column on the shape of a chromatogram peak. FIG. 8A shows the peak shape of a chromatogram peak at various elution injection volumes in the absence of an affinity chromatography material to interact with. FIG. 8B shows the peak shape of a chromatogram peak at various elution injection volumes in the in the presence of an affinity chromatography material.

### DETAILED DESCRIPTION

Disclosed herein are single injection elution methods for use with affinity chromatography techniques and systems. In order that the methods and technology may be more readily understood, certain terms are first defined. In addition, it should be noted that whenever a value or range of values of a parameter are recited, it is intended that values and ranges intermediate to the recited values are also part of this disclosure. The word about, if not defined otherwise, means±5%. It is also to be noted that as used herein and in the claims, the singular forms “a” and “the” include plural references unless the context clearly dictates otherwise.

### Definitions

As used herein, the term “conjugate” refers to the linkage of two molecules formed by the chemical bonding of a reactive functional group of one molecule with an appropriately reactive functional group of another molecule. Nonporous polymer particles may have one or more affinity reagents conjugated to the surface of said particles. For example, one or more affinity agents, such as Protein A, may be conjugated directly to a particle via an interaction with an epoxide on the surface of the particle. Alternatively, an affinity agent may be indirectly conjugated to the surface of the nonporous polymer particles via a linker (such as a polyethylene glycol (PEG) linker) or via an interaction with a streptavidin molecule. For the latter instance, one or more streptavidin molecules are conjugated directly to a particle via an interaction with an epoxide on the surface of the particle, which can then bind, via an ionic interaction, to a biotinylated affinity agent.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)₂, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)₂ fragments refer to antibody fragments that lack the Fc portion of an intact antibody.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein, the term “bispecific antibody” refers to an antibody that is capable of binding at least two different antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

### Affinity Chromatography Systems, Columns, and Materials

### Particles

In one aspect, the present technology utilizes affinity chromatography columns for the purification and isolation of a target analyte. The affinity chromatography columns are suitable for use in a high-performance liquid chromatography (HPLC) system or an ultra-high performance liquid chromatography (UHPLC) system and are designed for robust on-column affinity capture at the high pressures and flow conditions of said systems.

The affinity chromatography columns used in the methods disclosed herein comprise nonporous particles, which provide high surface area for conjugation of affinity agents and can withstand the pressures of HPLC and UHPLC systems. As such, in one aspect the affinity chromatography columns comprise a plurality of nonporous particles having an average particle size between 1.0 μm to 10 μm. In a preferred embodiment, the nonporous particles are nonporous polymer particles. In some embodiments, each particle within the plurality of particles is highly spherical with a smooth surface. In some embodiments, each particle within the plurality of particles is highly spherical with a bumpy, convex surface. Such materials have surface areas (measured in m²/g) that are close to their theoretical values. The theoretical surface area for a nonporous smooth sphere is equal to 6/{particle diameter×particle density}. For example, 1 micron polymer particles with a density of approximately 1 g/mL has a theoretical surface area of 6 m²/g, a 3.5 micron polymer particle with the same density has a theoretical surface area of 1.7 m²/g, and a 7 micrometer polymer particle with same density has a theoretical surface area of 0.9 m²/g.

The particles for use in the methods described herein are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). Without wishing to be bound by theory, it is believed that the use of nonporous particles is advantageous as it removes diffusion of analytes into pores of the particles, thereby by improving kinetics of the binding and eluting steps of affinity chromatography.

The nonporous particles described herein have an average particle size of between 1 to 10 microns. In some embodiments, the particle size is about 1.7 microns. In some embodiments, the particle size is about 3.5 microns. In some embodiments, the particle size is about 7 microns.

The size (i.e., less than 10 microns), shape (i.e., spherical), and surface area (i.e., nonporous smooth or nonporous bumpy convex) create a form factor useful for affinity chromatography and affinity chromatography columns used in conjunction with HPLC and UHPLC systems. These systems operate under high pressures (e.g., typically greater than 3,000 psi, such as, for example, 5,000 psi, 10,000 psi, 12,000 psi, 15,000 psi and so forth). Therefore, the particles used herein are rigid particles such that the form factor is retained under HPLC and UHPLC operating conditions.

As used herein, the term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Materials that meet the form factor requirements for forming a core (e.g., center or base) of the particles used herein include polymers, in particular organic polymers. Thus, in some embodiments, the nonporous particles include a nonporous polymer core. In some embodiments, the nonporous polymer core is divinylbenzene (DVB), for example divinylbenzene 80%. In some embodiments, the nonporous polymer core is formed to include two or more polymers. For example, in some embodiments the nonporous polymer cores include both DVB and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783, incorporated herein by reference.

Other nonporous materials can be utilized as long as the form factor of the particles can be maintained under the operating conditions of HPLC or UHPLC. That is, other materials, such as silica, metal oxides, hybrid inorganic-organic materials, or combinations thereof may be used to create nonporous spherical particles having an average particle size of less than 10 microns and the rigidity to retain form factor under high operating pressures (e.g., greater than 3000 psi).

### Affinity Agents

To form particles useful for affinity chromatography, the outer surface of the nonporous particle is conjugated, either directly or indirectly, to an affinity agent.

In embodiments for direct conjugation, the outer surface of the nonporous polymer core comprises a hydrophilic surface, such as, for example, an epoxide. One or more affinity agents can be directly conjugated to the hydrophilic surface. They hydrophilic surface (or hydrophilic layer, used interchangeably herein) can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (cpihydrochlorin), or ether formation (direct attachment of PEG or carbohydrate groups by ether formation). Further examples include (3-glycidyloxypropyl) trimethoxysilane, (3-glycidyloxypropyl)triethoxysilane, polyacrylate, poly(methyl acrylate), and combinations thereof. Additionally or alternatively, these may include glycidol, glyceroltriglycidyl ether, and combinations thereof.

An affinity agent can be directly conjugated to the hydrophilic surface of the nonporous particle via linkers and methods known in the art, and described in Hermanson G, “Bioconjugate Techniques” 3^{rd} Edition, July 2013).

In embodiments for indirect conjugation, one or more streptavidin molecules are first directly conjugated to the hydrophilic surface of the nonporous particles as described above. Due to the strong affinity between biotin and streptavidin, the streptavidin molecules provide a binding site for biotinylated affinity agents, providing a functionalized particle with a specific affinity (based on the affinity of the affinity agent).

Various affinity agents are suitable for use in the disclosed methods. These include immunoglobulin-binding proteins, antibodies or antigen-binding fragments thereof, oligonucleotides and nucleic acids, or other ligand-binding proteins or peptides. In embodiments wherein the affinity agent is indirectly conjugated to the particle, the affinity agent must be biotinylated.

### Immunoglobulin-binding Affinity Agents

In one aspect, the affinity agent is an immunoglobulin-binding protein. The immunoglobulin-binding protein provides accessible binding sites for an immunoglobulin, i.e., an antibody, provided that said antibody comprises a conserved region that binds to the immunoglobulin-binding protein. In one embodiment, the immunoglobulin-binding protein is Protein A. In another embodiment, the immunoglobulin-binding protein is Protein G. In other embodiments, the immunoglobulin-binding protein is Protein A/G or Protein L. In some embodiments, the immunoglobulin-binding protein is directly conjugated to the surface of the nonporous particle. In some embodiments, the immunoglobulin-binding protein is indirectly conjugated to the surface of the nonporous particle. In embodiments wherein the immunoglobulin-binding protein is indirectly conjugated to the surface of the nonporous particle, the immunoglobulin-binding protein is a biotinylated immunoglobulin-binding protein. For example, a biotinylated Protein A, a biotinylated Protein G, a biotinylated Protein A/G, or a biotinylated Protein L.

Most immunoglobulins (Ig) consist of four polypeptide chains: two identical heavy chains and two identical light chains that are connected by disulfide bonds. Within a given heavy chain or light chain, there is both a variable and a constant region. The constant region, which comprises 2-4 constant domains (depending on isotype), is highly conserved within a given isotype. As such, immunoglobulin-binding proteins that bind to a portion of the constant region are suitable for affinity capture of antibodies independent of the antibody's target antigen.

Immunoglobulin-binding proteins suitable for use in the present technology may exhibit strong binding affinity to the Fc portion of an antibody. This binding affinity can vary in strength by both isotype and species. For example, Protein A exhibits strong binding affinity to IgG isotypes but variable to no binding affinity to IgA, IgD, IgE, and IgM isotypes. Even within the IgG isotype, different subclasses can exhibit varied binding affinity. Protein A has high binding affinity to human IgG1, IgG2, and IgG4, but very weak binding affinity to IgG3. By contrast, Protein A binds to murine IgG3 but not to IgG1. Other examples of immunoglobulin-binding proteins, such as Protein G, have high binding affinity to all four subclasses of IgG. Methods for characterizing protein-protein interactions, including binding affinities across a range of environmental conditions, are well known in the art.

### Antibody Affinity Agents

In one aspect, the affinity agent is an antibody or antigen-binding fragment thereof. In some embodiments, the antibody or antigen-binding fragment thereof is a polyclonal antibody, a monoclonal antibody, a single-chain variable fragment (scFv), a nanobody, a monobody, a single domain antibody, a bispecific antibody, or a camelid. In some embodiments, the antibody or antigen-binding fragment thereof is an IgG, IgM, IgA, IgE, or IgD isotype. The antibody or antigen-binding fragment thereof may be derived from a human, mouse, rabbit, goat, or other species. In some embodiments, the antibody is a humanized antibody. In yet other embodiments, the antibody or antigen-binding fragment thereof is a biotinylated antibody or antigen-binding fragment thereof. That is, the biotinylated antibody or antigen-binding fragment thereof is a biotinylated polyclonal antibody, a biotinylated monoclonal antibody, a biotinylated scFv, a biotinylated nanobody, a biotinylated monobody, a biotinylated single domain antibody, a biotinylated bispecific antibody, or a biotinylated camelid.

In some embodiments, the affinity agent is an antibody or antigen-binding fragment thereof that specifically binds to insulin. In some embodiments, the antibody or antigen-binding fragment there of that specifically binds to insulin is a biotinylated antibody or antigen-binding fragment thereof. In some embodiments, the affinity agent is an antibody or antigen-binding fragment thereof that specifically binds to AAV9. In some embodiments, the antibody or antigen-binding fragment there of that specifically binds to AAV9 is a biotinylated antibody or antigen-binding fragment thereof. In some embodiments, the affinity agent is an antibody or antigen-binding fragment thereof that specifically binds to AAV2. In some embodiments, the antibody or antigen-binding fragment there of that specifically binds to AAV2 is a biotinylated antibody or antigen-binding fragment thereof. In some embodiments, the affinity agent is an antibody or antigen-binding fragment thereof that specifically binds to an AAV capsid, such as AAV1, AAV2, AAV3, AAV4, AAV5, AAV6, AAV7, AAV8, AAV9, AAVrh10 or a synthetic serotype thereof. In some embodiments, the antibody or antigen-binding fragment thereof that specifically binds to an AAV capsid is a biotinylated antibody or antigen-binding fragment thereof.

In some embodiments, the affinity agent is an antibody or antigen-binding fragment thereof that specifically binds to tacrolimus. In some embodiments, the antibody or antigen-binding fragment there of that specifically binds to tacrolimus is a biotinylated antibody or antigen-binding fragment thereof. In some embodiments, the affinity agent is an antibody or antigen-binding fragment thereof that specifically binds to troponin. In some embodiments, the antibody or antigen-binding fragment there of that specifically binds to troponin is a biotinylated antibody or antigen-binding fragment thereof. In some embodiments, the affinity agent is an antibody or antigen-binding fragment thereof that specifically binds to IgG. In some embodiments, the antibody or antigen-binding fragment there of that specifically binds to IgG is a biotinylated antibody or antigen-binding fragment thereof. In some embodiments, the affinity agent is an antibody or antigen-binding fragment thereof that specifically binds to a cytokine. In some embodiments, the antibody or antigen-binding fragment there of that specifically binds to a cytokine is a biotinylated antibody or antigen-binding fragment thereof. In some embodiments, the affinity agent is an antibody or antigen-binding fragment thereof that specifically binds to perfluoroalkyl substances (PFAS). In some embodiments, the antibody or antigen-binding fragment there of that specifically binds to PFAS is a biotinylated antibody or antigen-binding fragment thereof. In some embodiments, the affinity agent is an antibody or antigen-binding fragment thereof that specifically binds to a host cell protein (HCP). In some embodiments, the antibody or antigen-binding fragment there of that specifically binds to a HCP is a biotinylated antibody or antigen-binding fragment thereof.

As used herein, the term “host cell protein” refers to process-related proteinaceous impurities present in a host cell culture or host cell line used during biopharmaceutical manufacturing and production.

### Oligonucleotide and Nucleic Acid Affinity Agents

In another aspect, the affinity agent is an oligonucleotide, nucleic acid, or oligomer. In some embodiments, the oligonucleotide can range from 5-50 nucleotides. In some embodiments, the nucleotide comprises 25 nucleotides. Any or all of the nucleotides in a particular oligonucleotide or nucleic acid species can further be modified using methods known in the art, including biotinylating of the oligonucleotide or nucleic acid species. In particular, an oligonucleotide can be biotinylated on the 5′ or 3′ end.

Oligonucleotides suitable as affinity agents may be presented by the following Formula I:

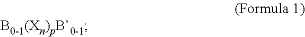

wherein B and B′ are independently a biotin group,
X is independently a nucleotide, nucleoside, or derivative thereof, including but not limited to adenosine, thymidine, guanosine, and cytidine;
n is independently 1-50; and
p is independently 1-50.

In some embodiments of Formula I, at least one of B or B′ is present (i.e., B or B′ is 1). In some embodiments, B is 1 and B′ is 0. In some embodiments, B is 0 and B′ is 1. In some embodiments, both B and B′ are 1. In some embodiments, both B and B′ are 0.

In some embodiments of Formula I, B is 1, X is thymidine, n is 25, p is 1, and B′ is 0. In some embodiments of Formula I, B is 0, X is thymidine, n is 25, p is 1, and B′ is 1. The resultant 5′-biotinylated or 3′-biotinylated oligonucleotide comprises 25 thymidine units (i.e., a 25-mer of thydine or dT₂₅).

In some embodiments, the affinity agent is a biotinylated oligonucleotide of Formula I. In some embodiments, the biotinylated oligonucleotide sequence is complementary to a target analyte sequence.

In one aspect, any nucleic acid-based affinity agent can be used, including biotinylated nucleic acid affinity agents and oligonucleotide affinity agents. The oligonucleotide may comprise deoxyribonucleic acids (DNA), ribonucleic acids (RNA), or a combination thereof. DNA oligonucleotides comprise the nucleotides cytidine, guanosine, adenosine, and thymidine. RNA oligonucleotides comprise the nucleotides cytidine, guanosine, adenosine, and uridine. In some embodiments, the oligonucleotide may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Oligonucleotides may further be modified at the nucleobase, sugar, or phosphodiester backbone with an array of chemical modifications which are further reviewed in Epple et al., Emerg. Top. Life. Sci. (2021) 5(5):691-697. In yet other embodiments, the nucleic acid affinity agent is an aptamer having specificity to a target analyte. Methods of biotinylating oligonucleotides, including modified oligonucleotides or oligonucleotides comprising non-naturally occurring nucleic acid analogues, are well known in the art and would be readily understood by a person of ordinary skill.

### Columns and Chromatography Systems

The materials, i.e., the nonporous particles conjugated to an affinity agent, are typically packed into a chromatographic device, such as a chromatographic column, thereby resulting in an affinity chromatography column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in US Patent Publication No. 2019/0086371 and US Application Publication No. 2022/0118443.

### Single Injection Elution Methods

Disclosed herein are methods of eluting a target analyte from an affinity chromatography column. In particular, the single injection elution method provided herein affords a concentrated, isolated sample by using a single injection of an elution buffer.

As used herein, “single injection of an elution buffer” (also described as a “single injection elution”) refers to a one-time injection of a specific volume of an elution buffer, for example, a single injection of 1 μL (or 10 μL, 20 μL, 30 μL, 40 μL, 50 μL, or any volume between 1-50 μL) of an elution buffer into the affinity chromatography column. In other embodiments, the single injection of an elution buffer has a volume that is between 50-100% of the column volume. For example, with a column having a column volume of 1 mL, the single injection of an elution buffer may be between 500 μL to 1 mL, or any volume in between (e.g., 600 μL, 700 μL, 800 μL, 900 μL). The single injection of an elution buffer can be achieved by injecting the elution buffer into the flow path of the affinity chromatography column using a sample injector. Alternatively, a switch valve upstream of the affinity chromatography column can be used to perform the single injection of elution buffer. The single injection is performed such that the volume of elution buffer bypasses or otherwise does not flow through a mobile phase mixer of a liquid chromatography system.

In contrast to the single injection elution, a gradient elution refers to the addition of elution buffer to the mobile phase over a period of time, such that the elution buffer mixes with the mobile phase buffer, thereby forming a gradient of the elution buffer (ranging from 0-100% of elution buffer). A gradient elution can vary in time, for example between 0.01 minutes to 3 minutes. The gradient is typically formed via a mobile phase mixer of a liquid chromatography system. Thus, and in contrast to the single injection methods described herein, when a gradient elution is performed in a very short period of time (e.g., 0.01-0.05 minutes), the theoretically sharp gradient change is distorted due to the mobile phase mixer of the liquid chromatography system.

As shown in Examples 1-7, the single injection of the elution buffer affords robust and fast elution of a target analyte from the affinity chromatography columns described herein. In particular, the single injection elution method described herein affords >50% recovery levels of a target analyte in less than 2 minutes. When using the single injection elution method, the target analyte elutes from the affinity chromatography column with sharp, narrow peaks. In some embodiments, the target analyte elutes from the affinity chromatography column having a peak width of between 1-10 seconds. Thus, the single injection elution method affords an eluted sample that is highly concentrated for the target analyte (i.e., in a small volume of elution buffer), enabling downstream analysis without further sample manipulation (e.g., dialysis, lyophilization, or other methods for concentrating a target analyte). Furthermore, the single injection elution allows for the direct elution of a target analyte from the affinity chromatography column to a detector without additional manipulation of the eluent.

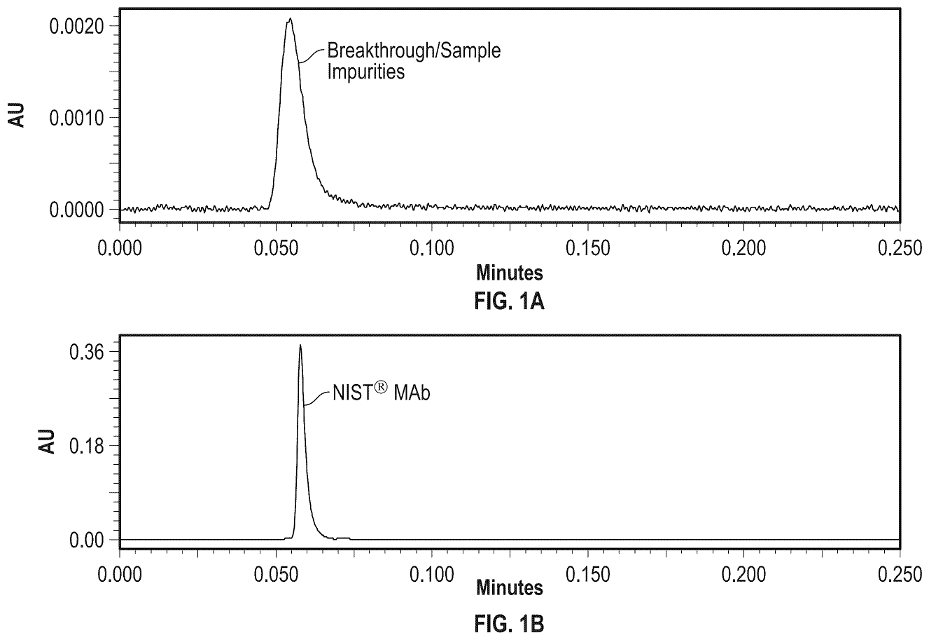

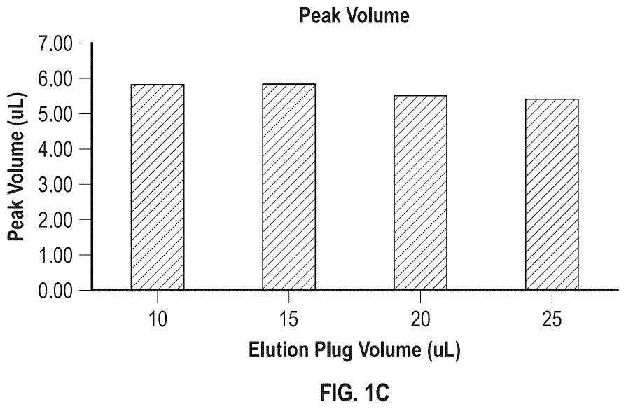

Example 1 and corresponding FIGS. 1A-1C, 2A-2B, and 3A-3B demonstrate that the single injection elution method can rapidly purify a monoclonal antibody using an elution time of less than 0.1 minutes. Thus, the single injection elution method is faster than the standard gradient elution method and affords higher sensitivity and sample detection.

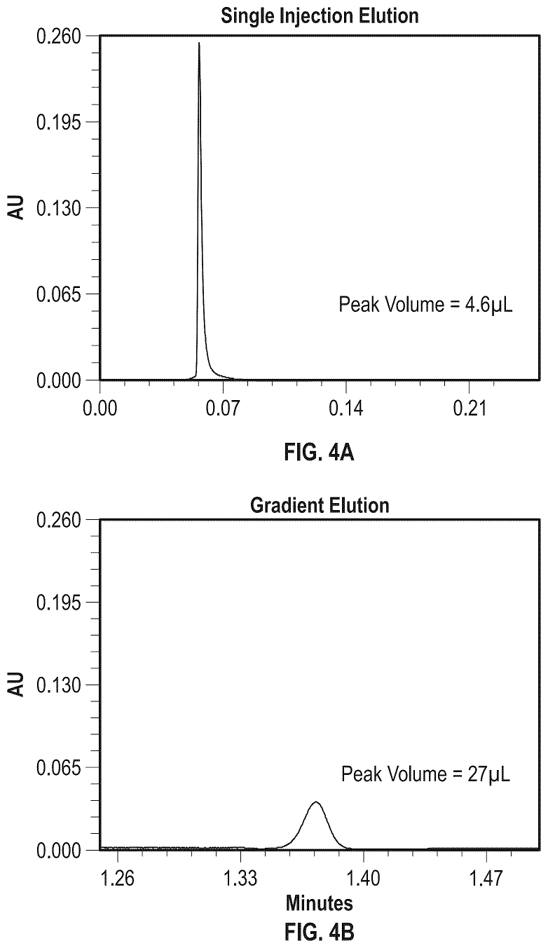

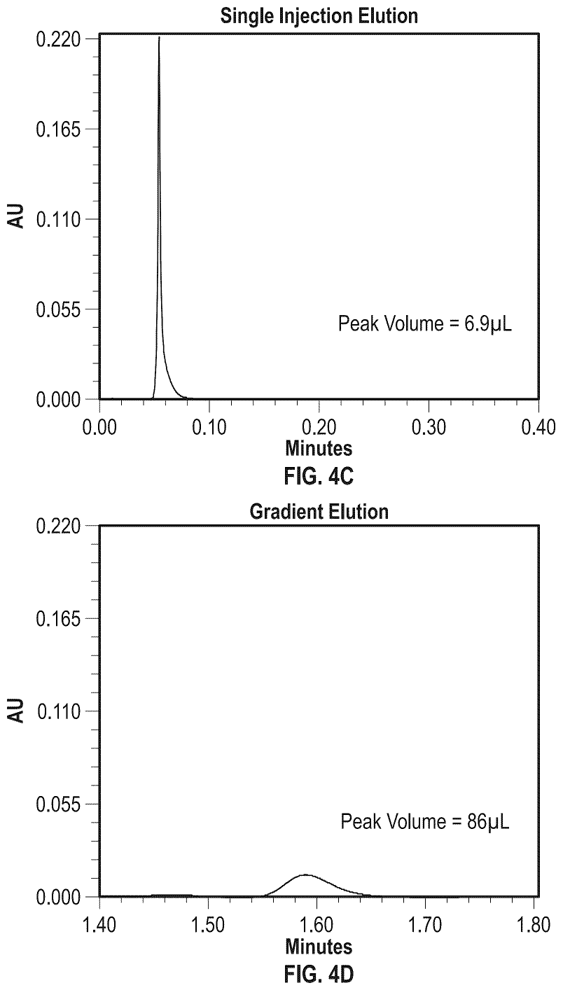

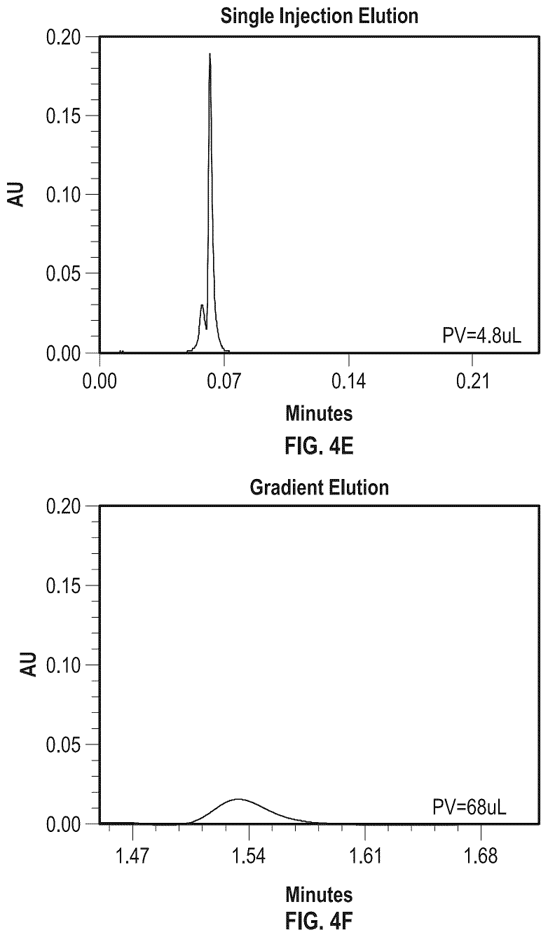

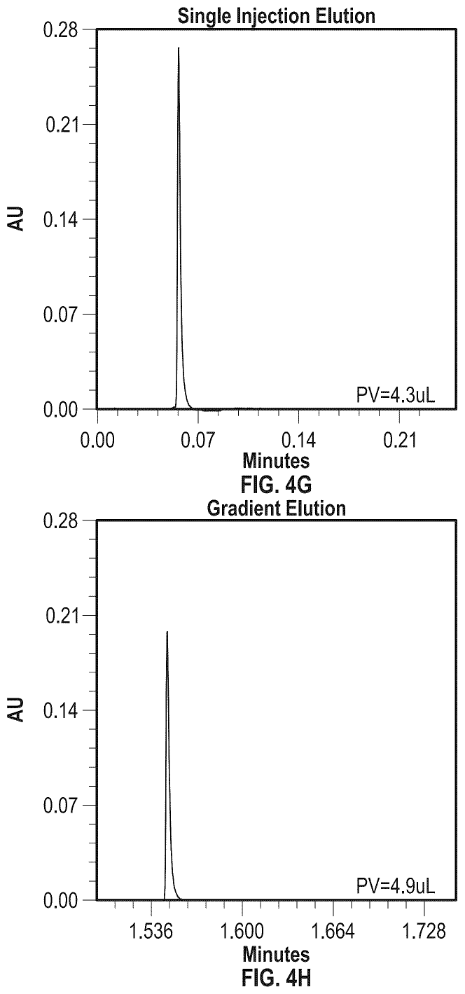

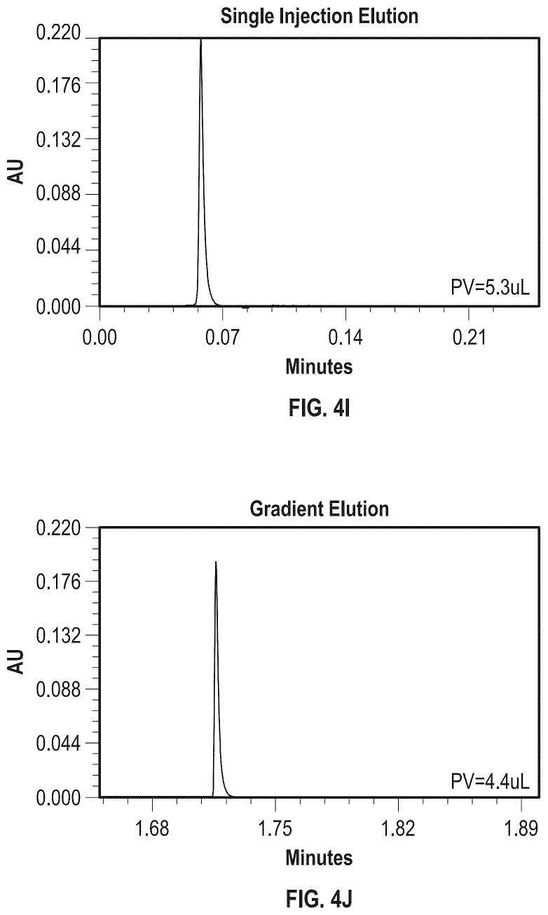

Example 2 and corresponding FIGS. 4A-4J demonstrate that the single injection elution method can be used with MS-compatible buffers, resulting in improved peak height and small peak volumes.

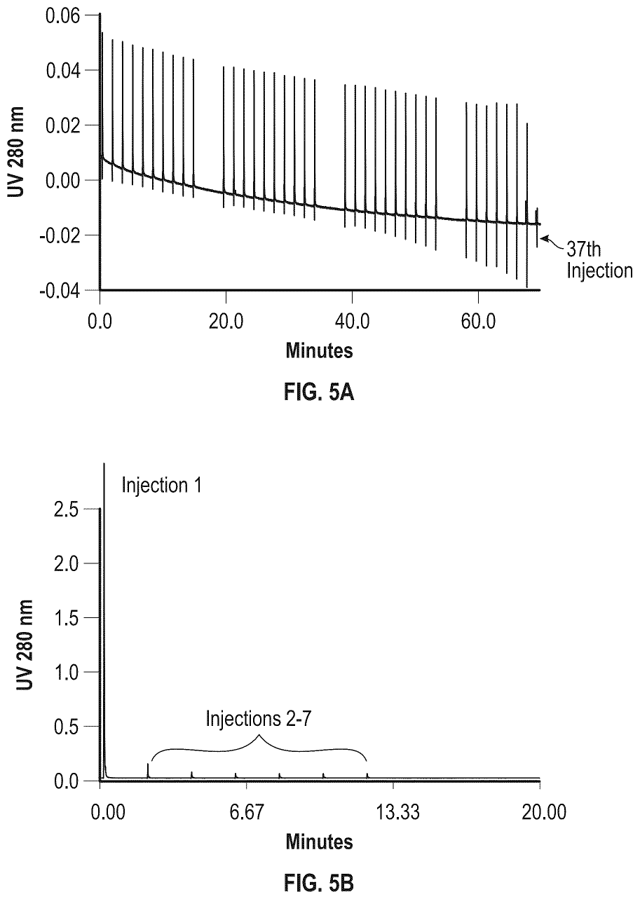

Example 3 and corresponding FIGS. 5A-5B demonstrate that the single injection elution method results in high recovery levels and enrichment of AAV capsids.

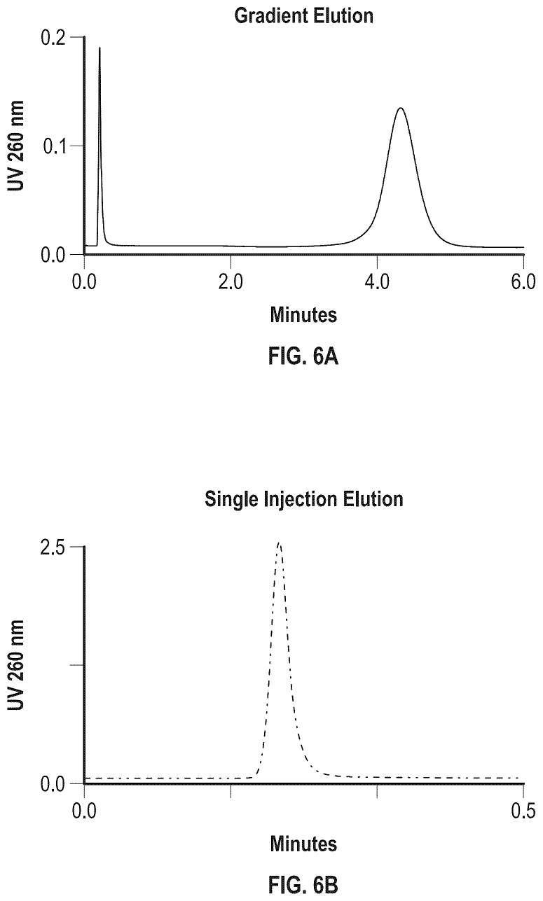

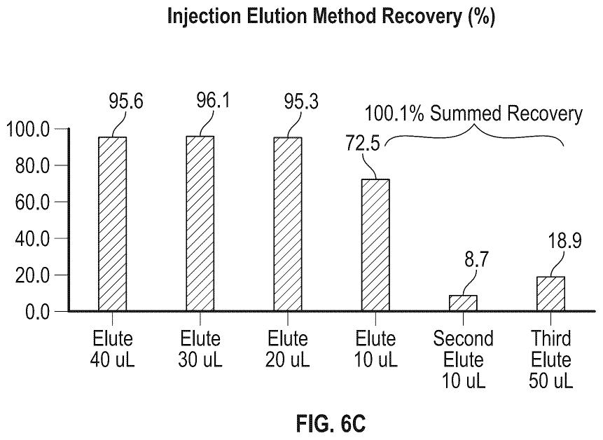

Example 4 and corresponding FIGS. 6A-6C demonstrate that the single injection elution method results in high recovery levels and enrichment of poly-A tailed mRNA.

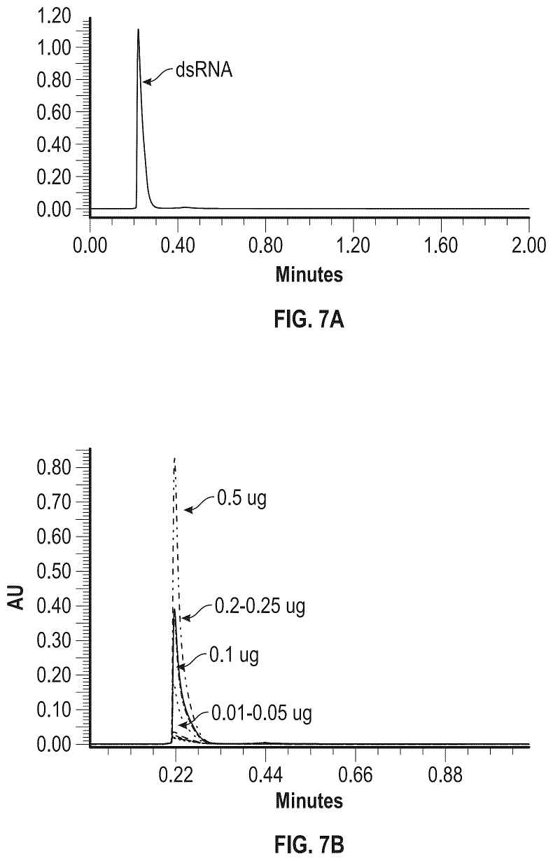

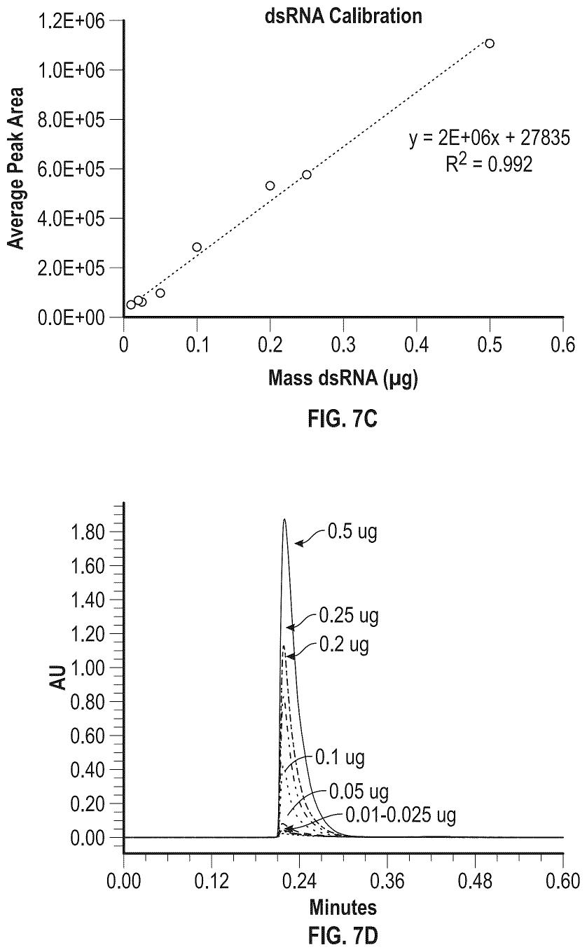

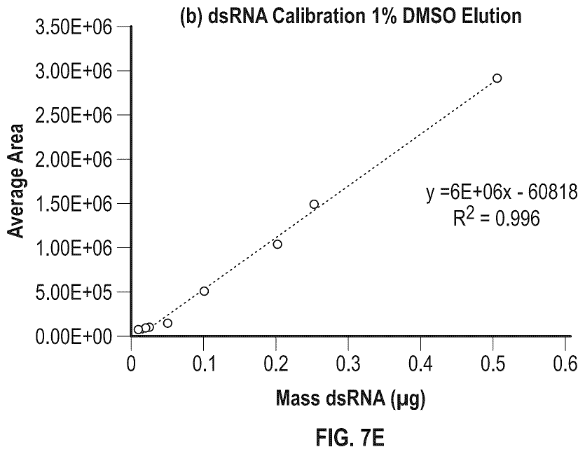

Example 5 and corresponding FIGS. 7A-7C demonstrate that the single injection elution method results in high recovery levels and enrichment of dsRNA.

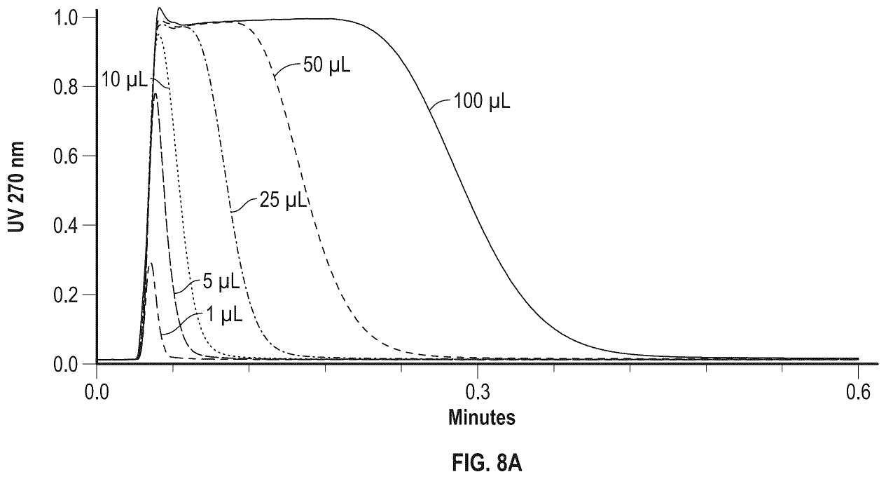

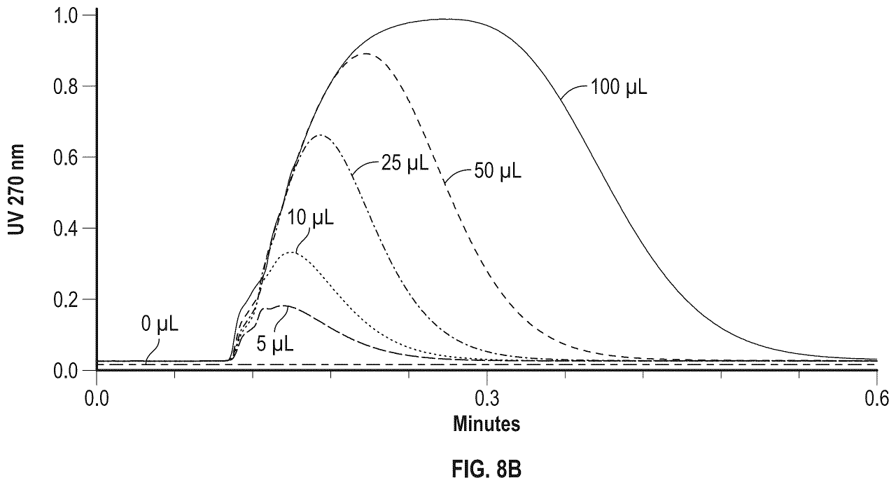

Example 6 and corresponding FIGS. 8A-8B demonstrate the dispersion of an injection buffer throughout the mobile phase, and the impact of injection volume on this dispersion.

Example 7 describes an exemplary method of directly eluting from an affinity chromatography column to a detector, without any intermediate manipulation of an analyte between the affinity chromatography column and the detector.

In some embodiments, the single injection of an elution buffer can be repeated 1, 2, 3, 4, or more times. In some embodiments, the single injection of the elution buffer is between 1 to 50 μL. In some embodiments, the single injection of the elution buffer is about 1 μL, about 10 μL, about 20 μL, about 30 μL, about 40 μL, or about 50 μL. In some embodiments, the single injection volume is at or greater than the void volume of the affinity chromatography column being used. In some embodiments, the single injection of the elution buffer is between 50-100% of the column volume of the affinity chromatography column.

In some embodiments, the single injection of an elution buffer is sufficient to result in at least 50% recovery of the target analyte. As used herein, the term “recovery” or “percent recovery” refers to the amount of target analyte in the elution volume as compared to the amount of target analyte in the initially loaded sample. Methods of calculating percent recovery are known in the art and would be understood by a skilled artisan. For example, percent recovery can be determined relative to a peak area that is known to include 100% of the loaded sample.

A number of elution buffers are suitable for use with the single injection elution methods described herein. These buffers include, but are not limited to, hydrochloric acid, trifluoroacetic acid, difluoroacetic acid, formic acid, acetic acid, or phosphoric acid. In some embodiments, the elution buffer has a pH of between 0.9 to 3.5. In some embodiments, the elution buffer has a pH of between 0.9 to 1.1, 1.1 to 1.3, 1.3 to 1.5, 1.5 to 1.7, 1.7 to 1.9, 1.9 to 2.1, 2.1 to 2.3, 2.3 to 2.5, 2.5 to 2.7, 2.7 to 2.9, 2.9 to 3.1, 3.1 to 3.3, or 3.3 to 3.5. In some embodiments, the elution buffer is compatible with mass spectrometry analysis. That is, the eluted sample can be directly analyzed using a mass spectrometer without the need for buffer exchange or dialysis. Additional elution buffers and concentrations thereof are described in the examples below.

The affinity chromatography columns, following the loading of a sample comprising the target analyte, may be washed to remove compounds present in the sample that are not bound by the affinity agent. A number of wash buffers suitable for use in liquid chromatography are known in the art and could be determined by a person of ordinary skill in the art. In some embodiments, the wash buffer has a pH of between 6.0 to 8.0. In some embodiments, the wash buffer has a pH of between 6.0 to 6.5, 6.5 to 7.0, 7.0 to 7.5, or 7.5 to 8.0. In some embodiments, the wash buffer comprises sodium phosphate, ammonium acetate, or ammonium formate. For example, but not by way of limitation, a suitable wash buffer may be 50-100 mM sodium phosphate, pH 7.2 to 7.4 or 100-200 mM ammonium formate pH 6.5. Additional washing buffers and concentrations thereof are described in the examples below.

### Detectors

In some embodiments a method of the present disclosure include detecting the target analyte with a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series. In some embodiments, the detector includes a high sensitivity detector (e.g., a mass spectrometer).

In some embodiments, a target analyte may be manipulated after elution from the affinity chromatography column and prior to detection with the detector. Traditionally, analytical affinity in combination with a high sensitivity detector produce a significant volume of eluent. If the entirety of the eluent were to flow directly to the detector, the signal would saturate the detector, rendering the detection method ineffective. To counteract this, the target analyte is manipulated in some way in order to send an appropriate volume of the target analyte to the detector. For example, the target analyte may be isolated from the eluent of the affinity chromatography column and manually injected into the detector. As a further example, the chromatography system may include a valve (e.g., a switching valve or a splitting valve) downstream of the affinity chromatography column. The valve may be configured to split a small volume of the eluent (i.e., a portion of the eluent including the target analyte) to a different flow path in fluid communication with a detector.

Alternatively, a method of the present disclosure may include eluting directly from the affinity chromatography column to a detector. In contrast to traditional methods, methods of the present disclosure include an injection elution plug. By the nature of the experiment, these injection elution plugs are typically small volume (e.g., from about 1 μL to about 50 μL, e.g., about 1 μL, about 5 μL, about 10 μL, about 15 μL, about 20 μL, about 25 μL, about 30 μL, about 40 μL, about 50 μL, etc.). Therefore, injection elution methods are particularly well suited to direct analysis by a detector (i.e., analysis without prior manipulation. In some embodiments, a chromatography system of the present disclosure includes a direct fluidic connection between an outlet of the affinity chromatography column and an inlet of the detector. In some embodiments, the direct fluidic connection between the outlet of the affinity chromatography column and the detector does not include any valves. In some embodiments, the detector in fluidic connection to the outlet of the affinity chromatography column is a high-sensitivity detector. In some embodiments, the high-sensitivity detector is a mass spectrometry (MS) detector, a multi angle light scattering (MALS) detector, a charged aerosol detector, an evaporative light scattering detector, a field flow fractionation detector, or a charge detection mass spectrometry (CDMS) detector.

### EXAMPLES

### Example 1: Single Injection Elution of a Monoclonal Antibody

The following example describes the purification of a monoclonal antibody (NIST® RM 8671 available from MilliporeSigma, referred to herein as NISTmAb) using a Protein A affinity chromatography column. The Protein A affinity chromatography column comprises protein A conjugated to a 3.5 μm divinylbenzene/polystyrene nonporous core particle having a hydrophilic layer on its outer surface. The particles were packed into a 2.1×20 mm column body and connected to a high pressure liquid chromatography system (the ACQUITY Premier system available from Waters Technologies Corporation, Milford MA) connected to a TUV detector. Detection was performed at 280 nm.

The column was equilibrated with 100 mM sodium phosphate (pH 7.2) for 10 minutes at a flow rate of 0.5 mL/min. After equilibration, 2 μL of the monoclonal antibody sample (˜0.25 μg/μL or 0.5 μg of antibody) was injected onto the column using a flow rate of 1 mL/min and effluent monitored. The mAb sample binds to the Protein A affinity agent in the column, whereas impurities or other components present in the sample that do not bind to Protein A flow through as shown in the peak in the chromatogram of FIG. 1A. To elute the mAb sample from the column, a 10 μL single injection of 120 mM hydrochloric acid was used. The single injection resulted in a sharp peak, corresponding to the mAb sample. Notably, the single injection elution was performed in under 0.1 minutes (6 seconds) as shown in FIG. 1B. The sample eluted in a small volume (˜5.8 μL) with minimal carryover between independent runs (<1% carryover).

The impact of the elution buffer volume was next determined. The above method was repeated using a 2 μL injection of 0.5 μg/μL NIST® MAb sample (˜1 μg of antibody) and with different volumes of the elution buffer for the single injection elution step, namely 10 μL, 15 μL, 20 μL, or 25 μL of 120 mM hydrochloric acid. As shown in FIG. 1C, peak volume was not impacted based on the single injection elution volume.

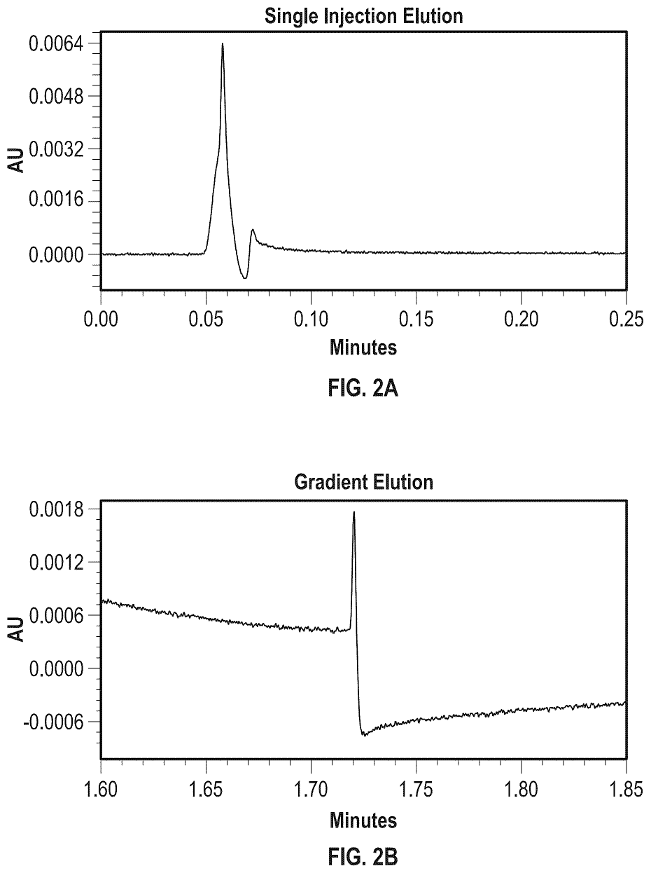

The sensitivity of the single injection elution method as compared to a gradient elution method was next determined, using 0.01 μg of the NIST® Mab sample (approximately 50 to 100× lower than the concentration used in FIGS. 1A-1C). For the single injection elution method, the sample was loaded using 100 mM sodium phosphate buffer (pH 7.2) and eluted with 10 μL of 120 mM HCl at a flow rate of 1 mL/min. For the gradient elution method, the sample was loaded using 100 mM sodium phosphate buffer (pH 7.2) and eluted with 12 mM HCl over 1 minute. The results for the single injection elution (FIG. 2A) and gradient elution (FIG. 2B) demonstrate that the single injection elution method is ˜3× more sensitive at low concentrations.

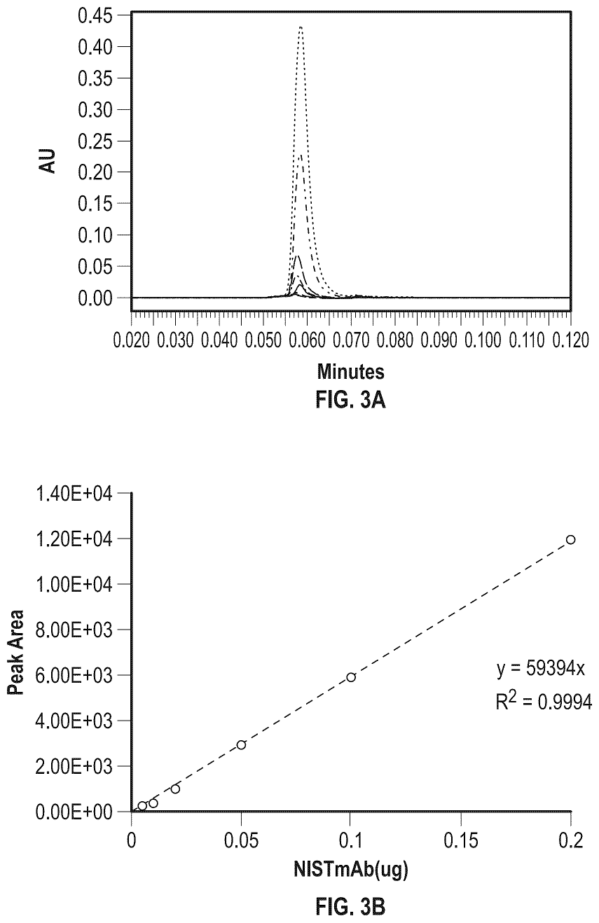

The single injection elution method was further tested for linearity, using sample concentrations of the NIST® MAb sample ranging from 0.001 to 0.5 μg/μL. Approximately 2 μL of the samples were loaded using 100 mM sodium phosphate (pH 7.2) and eluted with a single injection of 10 μL of 120 mM HCl (pH 0.92). As shown in FIG. 3A and FIG. 3B, the single injection elution method had a strong linear correlation across the concentrations tested.

### Example 2: Single Injection Elution with Mass Spectrometry (MS) Compatible Buffers

As demonstrated in the following example, an advantage of the single injection elution methods disclosed herein is that elution can be performed with MS-compatible buffers without sacrificing peak sensitivity or significantly increasing peak volume. The Protein A affinity chromatography column and NIST® MAb sample of Example 1 were used.

### Formic Acid Elution Buffer

Approximately 2 μL of 0.25 μg/μL NISTR MAb was loaded onto the column using 200 mM ammonium formate (pH 6.5) with a flow rate of 1 mL/min. For the single injection elution, a single injection of 10 μL of 500 mM formic acid was used. For the gradient elution, 500 mM formic acid over 1 minute was used. As shown in FIG. 4A (single injection elution) and FIG. 4B (gradient elution), the single injection elution method resulted in ˜ 7× higher peak height and ˜5.8× smaller peak volume as compared to the gradient elution method.

### Difluoroacetic Acid (DFA) Elution Buffer

Approximately 2 μL of 0.25 μg/μL NISTR MAb was loaded onto the column using 200 mM ammonium formate (pH 6.5) with a flow rate of 1 mL/min. For the single injection elution, a single injection of 10 μL of 10% DFA was used. For the gradient elution, 0.1% DFA over 1 minute was used. As shown in FIG. 4C (single injection elution) and FIG. 4D (gradient elution), the single injection elution method resulted in ˜16× higher peak height and ˜12× smaller peak volume as compared to the gradient elution method.

### Trifluoroacetic Acid (TFA) Elution Buffer

Approximately 2 μL of 0.25 μg/μL NISTR MAb was loaded onto the column using 200 mM ammonium formate (pH 6.5) with a flow rate of 1 mL/min. For the single injection elution, a single injection of 10 μL of 0.1% TFA was used. For the gradient elution, 0.1% TFA over 1 minute was used. As shown in FIG. 4E (single injection elution) and FIG. 4F (gradient elution), the single injection elution method resulted in ˜4× higher peak height and ˜14× smaller peak volume as compared to the gradient elution method.

### Phosphoric Acid Elution Buffer

Approximately 2 μL of 0.25 μg/μL NISTR MAb was loaded onto the column using 50 mM sodium phosphate (pH 7.4) with a flow rate of 1 mL/min. For the single injection elution, a single injection of 10 μL of 120 mM phosphoric acid was used. For the gradient elution, 24 mM phosphoric acid over 1 minute was used. As shown in FIG. 4G (single injection elution) and FIG. 4H (gradient elution), the single injection elution method resulted in ˜0.25× higher peak height and similar peak volume as compared to the gradient elution method.

### Hydrochloric Acid Elution Buffer

Approximately 2 μL of 0.25 μg/μL NISTR MAb was loaded onto the column using 50 mM sodium phosphate (pH 7.4) with a flow rate of 1 mL/min. For the single injection elution, a single injection of 10 μL of 120 mM HCl was used. For the gradient elution, 12 mM HCl over 1 minute was used. As shown in FIG. 4I (single injection elution) and FIG. 4J (gradient elution), the single injection elution method resulted in 14% higher peak height and similar peak volume as compared to the gradient elution method. Notably, the single injection elution method eluted the peak in under 0.07 minutes as compared to the >1 minute elution time for the gradient elution method.

Table 1 provides a summary of peak volumes that result from either a single injection elution or gradient elution method using the NIST® MAb sample with varying elution buffers and a 50 mM sodium phosphate (pH 7.4) binding buffer.

TABLE 1
Summary of Peak Volumes Resulting from Elution Methods

| Binding Buffer | Elution Buffer | Elution Method | Peak Volume (uL) |
|---|---|---|---|
| 50 mM Sodium | Acetic Acid | Single Injection | 6.1 |
| Phosphate (pH | (500 mM) | Gradient | 37.7 |
| 7.4) | Formic Acid | Single Injection | 4.3 |
| | (500 mM) | Gradient | 12.9 |
| | DFA (0.10%) | Single Injection | 4.9 |
| | | Gradient | 4.84 |
| | TFA (0.10%) | Single Injection | 5.0 |
| | | Gradient | 4.86 |
| | HCl (12 mM) | Single Injection | 5.3 |
| | | Gradient | 4.57 |
| | Phosphoric | Single Injection | 4.3 |
| | Acid (24 mM) | Gradient | 4.48 |

In sum, the single injection elution method disclosed herein affords fast elution with low peak volumes and allows for samples to be eluted in MS-compatible buffers. Therefore, the provided methods increase workflow efficiency and minimize subsequent sample manipulation prior to analysis.

### Example 3: Single Injection Elution of AAV Capsids

The following example describes the purification of AAV9 capsid particles using an anti-AAVX affinity chromatography column. The AAVX affinity chromatography column is a 2.1×20 mm column body comprising 3.5 μm divinylbenzene/polystyrene nonporous particles conjugated to streptavidin, wherein the streptavidin binding sites are occupied with a biotinylated monobody that specifically binds to AAV capsids, including AAV9. The 3.5 μm divinylbenzene/polystyrene nonporous particles within the affinity column have a hydrophilic layer on its outer surface. The column was connected to a high pressure liquid chromatography system (the ACQUITY Premier system available from Waters Technologies Corporation, Milford MA) connected to a TUV detector. Detection was performed at 280 nm.

The affinity chromatography column was loaded using 36×5 μL injections of AAV9 sample (˜10⁻¹³ viral particle/mL) or a total of 180 μL of sample. The sample was loaded using 0.1M sodium phosphate buffer at a 0.2 mL/min flow rate. The column reached saturation at the 37^{th} injection (see FIG. 5A). The AAV9 capsids were eluted using single injections of 10 μL of 1% phosphoric acid. As shown in FIG. 5B, approximately 70% of the particles were recovered in the first injection, representing an 18× enrichment of the sample. Subsequent injections of 10 μL of 1% phosphoric acid (injections 2-7) resulted in partial elution of the remaining AAV9 capsids retained on the column.

### Example 4: Single Injection Elution of Firefly Luciferase (FLuc) mRNA

The following example describes the purification of FLuc mRNA using a dT₂₅ affinity chromatography column. The dT₂₅ affinity chromatography column is a 2.1×20 mm column body comprising 3.5 μm divinylbenzene/polystyrene nonporous particles conjugated to streptavidin, wherein the streptavidin binding sites are occupied with a biotinylated dT₂₅ oligonucleotide. The 3.5 μm divinylbenzene/polystyrene nonporous particles within the affinity column have a hydrophilic layer on its outer surface. The column was connected to a high pressure liquid chromatography system (the ACQUITY Premier system available from Waters Technologies Corporation, Milford MA) connected to a TUV detector. Detection was performed at 260 nm.

To determine the elution profile with a gradient method, the affinity chromatography column was loaded with FLuc mRNA using 0.1M sodium phosphate buffer (pH 7.4) at a 0.2 mL/min flow rate and a 2-minute step gradient executed at 0 minutes (i.e., transition from 100% sodium phosphate buffer to 100% elution buffer (water) over 2 minutes). The first peak is poly (A) tail free mRNA that is not retained by the column and the mRNA peak eluted at 4.3 minutes with a peak volume of 217 μL (FIG. 6A).

To determine the elution profile with a single injection method, the affinity chromatography column was loaded with FLuc mRNA using 0.1M sodium phosphate buffer (pH 7.4) at a 0.2 mL/min flow rate. The mRNA was eluted using a single injection of 40 μL of water. As shown in FIG. 6B, the mRNA eluted with a sharper and more intense peak (>10× higher than the gradient method) and with a low peak volume (˜10.3 μL).

The effect of single injection elution volume was determined. FLuc mRNA was loaded onto the column as described above. A single injection elution volume of 40 μL, 30 μL, and 20 μL were tested, each of which resulted in ˜95-96% recovery of the total mRNA loaded (FIG. 6C). A single injection elution volume of 10 μL resulted in ˜70% recovery of the total mRNA loaded. A second single injection elution volume of 10 μL resulted in an additional 8.7% recovery. A third single injection elution volume of 50 μL resulted in the final 18.9% recovery-resulting in ˜ 100% recovery of the sample (FIG. 6C).

### Example 5: Injection Elution of Double-Stranded RNA (dsRNA)

The following example describes the purification of dsRNA using an anti-dsRNA affinity chromatography column. The anti-dsRNA affinity chromatography column is a 2.1×20 mm column body comprising 3.5 μm divinylbenzene/polystyrene nonporous particles conjugated to streptavidin, wherein the streptavidin binding sites are occupied with a biotinylated anti-dsRNA antibody (Antibody J2, available from Biotechne). The 3.5 μm divinylbenzene/polystyrene nonporous particles within the affinity column have a hydrophilic layer on its outer surface. The column was connected to a high pressure liquid chromatography system (the ACQUITY Premier system available from Waters Technologies Corporation, Milford MA) connected to a TUV detector. Detection was performed at 260 nm.

The affinity chromatography column was loaded with 2 μL of dsRNA 20KH (0.25 μg/μL concentration) using 100 mM sodium phosphate (pH 7.4) at a 0.2 mL/min flow rate. The dsRNA was eluted using a single injection of 50 μL of water. FIG. 7A shows the elution profile, which shows a sharp peak corresponding to the dsRNA sample. The single injection elution method afforded a fast elution time (less than 30 seconds).

The above method was repeated using different concentrations of KH20 dsRNA. dsRNA was loaded onto the column at 0.01 ug, 0.02 ug, 0.025 ug, 0.05 ug, 0.1 ug, 0.2 ug, 0.25 ug, or 0.5 ug using 100 mM sodium phosphate (pH 7.4) at a 0.2 mL/min flow rate. Sample was eluted using a single injection of 50 μL of water. FIG. 7B shows the elution profile, which shows sharp peaks corresponding to the dsRNA, particularly at the higher concentrations tested. The peak area and sample concentration exhibited a strong linear correlation (R² of 0.992) as shown in FIG. 7C. Thus, the single injection elution method with pure water affords fast elution times (less than 30 seconds), with sensitivity as low as 10 ng.

An additional elution buffer was tested using the different concentrations of KH20 dsRNA. dsRNA was loaded onto the column at 0.01 ug, 0.02 ug, 0.025 ug, 0.05 ug, 0.1 ug, 0.2 ug, 0.25 ug, or 0.5 ug using 100 mM sodium phosphate (pH 7.4) at a 0.2 mL/min flow rate. Sample was eluted using a single injection of 50 μL of 1% DMSO in water. FIG. 7D shows the elution profile, which shows sharp peaks corresponding to the dsRNA tested, particularly at the higher concentrations tested. The peak area and sample concentration exhibited a strong linear correlation (R² of 0.996) as shown in FIG. 7E. Thus, the single injection elution method with 1% DMSO in water affords fast elution times (less than 30 seconds) with sensitivity as low as 10 ng.

### Example 6: Impact of Elution Time on Peak Shape

This example is directed to determining the impact of the time an elution plug spends traversing a chromatography column (which itself is a function of, inter alia, flow rate and injection volume) on the resulting peak shape. FIG. 8A-8B shows the peak shape of an elution injection at an elution injection volume of 1 μL, 5 μL, 10 μL, 25 μL, 50 μL, and 100 μL. For this exemplary experiment, a mobile phase of pure MeOH, and an elution injection of 99:1 MeOH:acetone was used. The data is normalized such that an absorbance of 1 unit represents a solution with the maximum concentration of acetone. In other words, 1 unit of absorbance is equivalent to the expected absorbance of acetone in a 99:1 MeOH:acetone mixture. Similarly, an absorbance of less than 1 unit indicates the acetone has been diluted in the MeOH mobile phase, and therefore displays a locally decreased absorbance.

FIG. 8A highlights the dispersion of acetone throughout the mobile phase after the elution injection is applied to the mobile phase at a flow rate of 0.4 mL/min. In this experiment, there was no chromatography material within the column to impede flow of the elution injection. At time t=0, the elution injection was supplied. All experiments reached a peak (i.e., maximum expected acetone concentration) at around 0.06 min. At lower elution injection volumes (e.g., 1 μL, 5 μL, and 10 μL), the relative amount of acetone compared to the surrounding MeOH is insufficient to prevent rapid dispersion. As such, these elution injections never reach 1 unit of absorbance during elution. At 25 μL of elution injection volume, 1 unit of absorbance is reached, and maintained briefly (approximately 0.06 min), before dispersion reduces the local concentration of acetone to 0 absorbance by 0.15 min. Both 50 μL and 100 μL elution injection volumes also reach and maintain 1 absorbance unit of acetone (for about 0.12 min for 50 μL elution injection, and 0.18 min for 100 μL elution injection) before the acetone concentration is reduced. Notably, it takes significantly longer for the 100 μL elution injection to fall to 0 absorbance of acetone than any other elution injection (approximately 0.36 min).

FIG. 8B highlights the dispersion of acetone throughout a mobile phase interacting with a chromatography material. In this experiment, the mobile phase was supplied at 0.4 mL/min through a 2.1×20 mm chromatography column including a plurality of 3.5 μm particles with a hydrophilic coating. Notably, interaction with the column delays the peak onset in each experiment, such that they now all occur at different times (approximately 0.12 min for 5 μL elution injection, approximately 0.15 min for 10 μL elution injection, approximately 0.17 min for 25 μL elution injection, approximately 0.21 min for 50 μL elution injection, and approximately 0.24 min for 100 μL injection). Moreover, the only injection volume which achieves 1 absorbance unit of acetone is the 100 μL injection.

This data indicate that the full “strength” (i.e., the intended composition of an elution injection) is not maintained along a chromatography column for an elution injection of 1 μL, 5 μL, 10 μL, or 50 μL. Therefore, it is concluded that even though a 25 μL injection was capable of recovering 97% of the sample in Example 7, the maximum “strength” (the maximum possible ability for the elution injection to release the sample from the affinity chromatography column) of the elution injection was not present. This may in part provide an explanation for why 100% recovery was not observed for a single 25 μL injection in Example 7. Moreover, this data indicates that liquid chromatography systems subject to larger elution injection dispersion may in turn require larger elution injection volumes for efficient elution.

### Example 7: Direct Elution from an Affinity Chromatography Column to a Detector

The present example is directed to an exemplary chromatography system of the present disclosure wherein the eluent from an elution injection into an affinity chromatography column is passed directly to a detector without any intermediate manipulation of the eluent.

First, an affinity chromatography experiment is performed using an affinity chromatography column in fluid communication with a detector. The affinity chromatography column includes a plurality of nonporous particles within the column. The nonporous particles each have a hydrophilic layer their respective outer surfaces. The affinity chromatography material includes an affinity agent on a surface of the material (e.g., an immunoglobulin-binding protein, an antibody or antigen-binding fragment thereof, or an oligonucleotide). A sample (e.g., a sample including an immunoglobulin, an antigen, or an oligonucleotide) including a target analyte is then loaded onto the affinity chromatography column. The target analyte is retained by the affinity chromatography column. An elution injection is then supplied to the affinity chromatography column, thereby releasing the target analyte from the affinity chromatography column into the elution injection. The elution injection may be from about 1 μL to about 50 μL (e.g., about 1 μL, about 5 μL, about 10 μL, about 15 μL, about 20 μL, about 25 μL, about 30 μL, about 35 μL, about 40 μL, about 45 μL, about 50 μL, etc.). The target analyte then flows directly from an outlet of the affinity chromatography column to the detector. Due to the volume of the elution injection, the elution injection may then be detected at the detector without any further manipulation of the elution injection. The detector may be a mass spectrometry (MS) detector, a multi angle light scattering (MALS) detector, a charged aerosol detector, an evaporative light scattering detector, a field flow fractionation detector, or a charge detection mass spectrometry (CDMS) detector.

1. A method of purifying a target analyte, the method comprising:
a) loading a sample comprising the target analyte onto an affinity chromatography column, the affinity chromatography column comprising:
a plurality of nonporous polymer particles, wherein each particle within the plurality of nonporous polymer particles comprises a polymer core and a hydrophilic surface on an outer layer of the polymer core; and
one or more affinity agents conjugated directly to the hydrophilic surface of each particle within the plurality of nonporous polymer particles, or indirectly via an interaction with one or more streptavidin molecules on the hydrophilic surface of each particle within the plurality of nonporous polymer particles;
b) washing the affinity chromatography column with a wash buffer; and
c) eluting the target analyte from the affinity chromatography column using a single injection of an elution buffer, the single injection having a volume of between 1 μL to 50 μL.

2. (canceled)

3. The method of claim 1, wherein the affinity agent is an immunoglobulin-binding protein, an antibody or antigen-binding fragment thereof, or an oligonucleotide.

4. The method of claim 1, wherein the affinity agent is biotinylated.

5. The method of claim 3, wherein the immunoglobulin-binding protein is Protein A, Protein G, Protein A/G, Protein L, or a binding domain thereof.

6. The method of claim 3, wherein the antibody or antigen-binding fragment thereof binds to insulin, an AAV capsid, tacrolimus, troponin, IgG, a cytokine, a double-stranded RNA, a host cell protein, or perfluoroalkyl substances (PFAS).

7. The method of claim 6, wherein the AAV capsid is AAV1, AAV2, AAV3, AAV4, AAV5, AAV6, AAV7, AAV8, AAV9, AAVrh10, or a synthetic serotype thereof.

8. The method of claim 3, wherein the oligonucleotide is a poly-T oligonucleotide.

9. The method of claim 1, wherein nonporous polymer particles within the plurality of nonporous polymer particles have an average particle size between 1.0 μm to 10 μm.

10. The method of claim 1, wherein the wash buffer comprises sodium phosphate.

11. (canceled)

12. The method of claim 1, wherein the elution buffer comprises hydrochloric acid, trifluoroacetic acid, difluoroacetic acid, formic acid, acetic acid, or phosphoric acid.

13. (canceled)

14. The method of claim 1, wherein the elution buffer is water.

15. The method of claim 14, wherein the elution buffer further comprises dimethyl sulfoxide (DMSO).

16. (canceled)

17. The method of claim 1, wherein the single injection has a volume of about 1 μL, about 10 μL, about 20 μL, about 30 μL, about 40 μL, or about 50 μL.

18. (canceled)

19. The method of claim 1, further comprising step d) detecting the target analyte with a detector.

20. The method of claim 19, wherein the detector is an ultraviolet spectroscopy detector, a fluorescence spectroscopy detector, a mass spectrometry detector, a multi angle light scattering detector, an evaporative light scattering detector, a field flow fractionation detector, a charged aerosol detector, and/or a charge detection mass spectrometry detector.

21. The method of claim 19, wherein the elution buffer including eluted target analyte is passed directed to the detector without any intermediate manipulation.

22. The method of claim 1, wherein the eluting step c) is performed in less than 2 minutes.

23. (canceled)

24. The method of claim 1, wherein the eluting step c) results in a peak width of between 1 to 10 seconds.

25. The method of claim 1, wherein step c) is repeated.

26. The method of claim 1, wherein the eluting step c) results in at least 50% recovery of the target analyte.

27. (canceled)
